(19) United States
(12) Patent Application Publication
(10) Pub. No.: US 2006/0100287 A1
(43) Pub. Date: May 11, 2006

(54) Insulin-like growth factor-1 secretagogue

(76) Inventors: Kenji Okajima, Nagoya-shi (JP); Naoaki Harada, Kumamoto-shi (JP); Yuichi Teratani, Soraku-gun (JP); Urao Kawakami, Nabari-shi (JP); Shiho Bakoshi, Kashiba-shi (JP)

Correspondence Address: WENDEROTH, LIND & PONACK, L.L.P., 2033 K STREET N. W., SUITE 800, WASHINGTON, DC, 20006-1021, US

(21) Appl. No.: 11/176,266

(22) Filed: Jul. 8, 2005

(30) Foreign Application Priority Data
Nov. 5, 2004 (JP) 2004-322983

(51) Int. Cl. A61K 31/16

(52) U.S. Cl. 514/625

(57) ABSTRACT

The present invention relates to an IGF-1 secretagogue containing one or two or more compounds selected from capsaicinoids, capsinoids and synthetic capsaicin, which has no problem of skin irritation, etc., and is effective for, for example, reducing skin wrinkle and sagging skin by the increase of skin's thickness and elasticity, promoting hair growth.

### BACKGROUND OF THE INVENTION

1. Field of the Invention

The present invention relates to insulin-like growth factor-1 secretagogues containing capsaicinoids, capsinoids and/or synthetic capsaicin (hereinafter referred to as “capsaicinoid or the like” in some cases).

2. Background Art

A variety of skin external preparations containing capsaicin as a medicinal ingredient have been known. For, example, the followings are known: a skin external preparation containing capsaicin as an active ingredient for improving the action of body fluids, which is effective for beautification of the skin due to the action of body fluids, promoting moisture secretion from the dermis to the epidermis to retain sufficient moisture in the epidermis (c.f., for example, JP-A No. 1998-120558), a head skin pack prepared by addition of at least capsicum tincture, etc. to be a blended liquid with medium viscosity, which activates hair roots and increases scalp blood flow when applied to the scalp, thereby exerting excellent hair-growing and hair-nurturing effects (c.f., for example, JP-A No. 1997-12441), a hair-restoring cosmetic comprising capsaicin, etc. as an active ingredient, which has effects for restoring hair due to the blood flow-increasing effect (c.f., for example, JP-B No. 3435008), or a cosmetic for preventing skin roughness and wrinkles, yellowed hair, split ends hair, hair breakage and the like and essentially improving the skin and hair, which comprises crude drugs such as red pepper, etc. containing a component with blood flow-increasing effects (c.f., for example, JP-A No. 2003-55139). However, capsaicin mixed in those skin external preparations as medicinally active ingredients are used for the purpose of, for example, promoting perspiration by their hyperthermic action, stimulating water secretion from the dermis to the epidermis, or increasing blood flow by stimulation of the skin.

Insulin-like growth factor-1 (hereinafter abbreviated as IGF-1) is a peptide hormone with a molecular weight of about 7500 kDa having a structure and effects that are very similar to insulin (c.f., for example, Goodman & Gilman's Pharmacology Book, Lower Volume, Basis and Clinic in Drug Therapy—Tenth Edition; Chapter 61 Insulin, Pharmacology of Oral Hypoglycemic Agents and Endocrinology of Pancreas, 2003 p. 2144, Translation Supervisor; Takaori Shuji, Fukuda Hideomi, Akaike Akinori, Published by Tokyo Hirokawa Bookstore).

It is known that IGF-1 actively keeps cells in good condition by promoting cell differentiation and stimulating cell proliferation, etc. (c.f., for example, Conn K J et al., J. Biol. Chem., 1996, vol. 271, No. 46, p. 28853-28860; and Braham C et al., Dermatology, 2002, vol. 20, No. 4, p. 325-329), and prevents aging (c.f., for example, Roubenoff R et al., Am. J. Med, 2003, vol. 115, No. 6, p. 501-502). In the above literatures, however, there is no description, for example, that capsaicinoid or the like promotes secretion of IGF-1, nor any suggestion of the relation between capsaicinoid or the like and IGF-1.

### SUMMARY OF THE INVENTION

An object of the present invention is to provide IGF-1 secretagogues having no problem such as skin irritation, which are effective, for example, for alleviating skin wrinkle and sagging skin by the increase of skin's thickness and elasticity, and for promoting hair growth, and the like.

The present inventors conducted intensive studies on the aforementioned problems and, as a result, they found that insulin-like growth factor-1 significantly increased in the mouse skin to which a skin external preparation containing about 0.001 to 0.1% by mass of capsaicinoid or the like was applied. They carried out further researches and finally completed the present invention.

Thus, the present invention relates to:

(1) an insulin-like growth factor-1 secretagogue containing one or two or more compounds selected from capsaicinoids, capsinoids and synthetic capsaicin;

(2) the insulin-like growth factor-1 secretagogue as described in the above (1), wherein the compound is contained in an amount of 0.001 to 0.1% by mass;

(3) the insulin-like growth factor-1 secretagogue as described in the above (1) or (2), which promotes differentiation, proliferation and intercellular substance productivity of skin fibroblasts;

(4) the insulin-like growth factor-1 secretagogue as described in the above (3), wherein the intercellular substance is one or two or more members selected from the group consisting of skin collagen, skin elastin and skin hyaluronic acid;

(5) the insulin-like growth factor-1 secretagogue as described in any one of the above (1) to (4), wherein capsaicinoids contains at least one compound selected from the group consisting of (6E)-N-[(4-hydroxy-3-methoxyphenyl)methyl]-8-methyl-6-nonenamide, dihydrocapsaicin, nordihydrocapsaicin, homocapsaicin, and homodihydrocapsaicin;

(6) the insulin-like growth factor-1 secretagogue as described in any one of the above (1) to (4), wherein capsicinoids contain at least one compound selected from the group consisting of capsiate, dihydrocapsiate, nordihydrocapsiate, homocapsiate and homodihydrocapsiate;

(7) the insulin-like growth factor-1 secretagogue as described in any one of the above (1) to (4), wherein synthetic capsaicin is nonylic vanillylamide or decanoic acid vanillylamide;

(8) the insulin-like growth factor-1 secretagogue as described in the above (1), wherein capsaicinoids contain 55% by mass or more of (6E)-N-[(4-hydroxy-3-methoxyphenyl)methyl]-8-methyl-6-nonenamide;

(9) the insulin-like growth factor-1 secretagogue as described in the above (1), wherein capsaicinoids contains 98% by mass or more of (6E)-N-[(4-hydroxy-3-methoxyphenyl)methyl]-8-methyl-6-nonenamide;

(10) The insulin-like growth factor-1 secretagogue as described in any one of the above (3) to (9), which is a skin external preparation;

(11) the insulin-like growth factor-1 secretagogue as described in the above (10), which further contains one or two or more compounds selected from the group consisting of nonionic surfactant, amphoteric surfactant and cationic surfactant at a rate of 5% by mass or more, relative to the whole insulin-like growth factor-1 secretagogue;

(12) the insulin-like growth factor-1 secretagogue as described in the above (11), wherein the nonionic surfactant contains one or two ore more compounds selected from the group consisting of polyoxyethylene castor oil, polyoxyethylene hydrogenated castor oil, polyethylene glycol fatty acid ester, polyoxyethylene alkyl ether, polyoxyethylene polyoxypropylene alkyl ether, polyoxyethylene alkylphenyl ether and polyoxyethylene sorbitan fatty acid ester;

(13) the insulin-like growth factor-1 secretagogue as described in the above (11), wherein the amphoteric surfactant is an alkyldimethylamino acetic acid betaine;

(14) the insulin-like growth factor-1 secretagogue as described in the above (11), wherein the cationic surfactant is an alkyl ammonium salt;

(15) the insulin-like growth factor-1 secretagogue as described in any one of the above (10) to (14), which is in the dosage form of liquid preparation, suspension, emulsion, ointment, cream, gel, liniment, lotion, cataplasm or pack; and

(16) The insulin-like growth factor-1 secretagogue as described in the above (15), which comprises 0.001 to 0.1% by mass of capsaicinoids containing 98% by mass or more of (6E)-N-[(4-hydroxy-3-methoxyphenyl)methyl]-8-methyl-6-nonenamid and 5 to 18% by weight of polyoxyethylene hydrogenated castor oil.

The present invention also relates to a method to stimulate IGF-1 secretion in the skin by applying a skin external preparation containing about 0.001 to 0.1% by mass of one or two or more compounds selected from capsaicinoids, capsinoids and synthetic capsaicin to the skin of mammals. Furthermore, the present invention relates to the use of one or two or more compounds selected from capsaicinoids, capsinoids and synthetic capsaicin for producing a skin external preparation having IGF-1 secretion-promoting activity.

The IGF-1 secretagogue of the present invention can stimulate secretion of IGF-1, particularly IGF-1 in the skin. IGF-1 activates skin fibroblasts so that it promotes differentiation, proliferation and functions of the fibroblasts. Fibroblasts promote the production of intercellular substances in the body such as collagen, elastin, hyaluronic acid and the like. Collagen is the main protein of connective tissues, and skin collagen is involved in contour formation of the skin such as texture or elasticity of the skin. Elastin is, along with collagen, a fibrous protein existing in connective tissues in the skin, and has a function to retain elasticity, firmness and moisture of the skin by bundling collagen fibers, which support the skin. Hyaluronic acid is a kind of glucosaminoglucans existing abundantly in the connective tissues of mammals, which attracts moisture to the skin and functions as a cushion. For reasons mentioned above, the IGF-1 secretagogue of the present invention can increase IGF-1 in the skin, resulting in increase of collagen, elastin, and hyaluronic acid in the skin, and thus it can maintain elasticity, firmness and moisture of the skin. Moreover, if the IGF-1 secretagogue of the present invention is applied to the skin with decreased elasticity or wrinkles and sagging, secretion of IGF-1 is promoted at the application site, and thus the elasticity of the skin at the site is recovered, smoothing wrinkles and improving sagging skin.

Furthermore, the IGF-1 secretagogue of the present invention increases IGF-1 in the skin and promotes differentiation, proliferation and functions of fibroblasts, and thus it can speed healing of wounds, postoperative wounds, etc.

In addition, since IGF-1 activates cells in hair roots such as hair matrix cells and hair papilla cells, the IGF-1 secretagogue of the present invention can apply to scalp for the purpose of hair restoration or hair growth.

The skin external preparation of the present invention causes no skin irritation based on capsaicinoid or the like, and therefore it can be used safely for a long period of time.

### BEST MODE FOR CARRYING OUT THE INVENTION

Hereinafter, the best mode for carrying out the present invention will be described.

“Capsaicinoids” in the present invention are a group of compounds having a basic structure of vanillylamide, which are the pungent principle of Capsicum annuum L.

Examples of the capsaicinoids are (6E)-N-[(4-hydroxy-3-methoxyphenyl)methyl]-8-methyl-6-nonenamide (hereinafter abbreviated as capsaicin (C₁₈H₂₇NO₃)), dihydrocapsaicin, nordihydrocapsaicin, homocapsaicin, homodihydrocapsaicin or the like. A mixture of one or more members selected from the above compounds may be used as capsaicinoid. For example, a capsaicin listed in the Pharmacopoeia of the United States of America (in which capsaicinoids are contained in 90 to 110%: the content of capsaicin (C₁₈H₂₇NO₃) is 55% or more, and the sum total content of capsaicin (C₁₈H₂₇NO₃) and dihydrocapsaicinoid (C₁₈H₂₉NO₃) is 75% or more, and the content of other capsaicinoid is 15% or less); a capsaicin (C₁₈H₂₇NO₃) which is usually called capsaicin and is distributed in the market; a mixture of other capsaicinoids and the like may be used as the capsaicinoids of the present invention.

“Synthetic capsaicin” means a capsaicinoid-like synthetic compound having the above-mentioned vanillylamide structure. The synthetic capsaicin includes, for example, nonylic vanillylamide and decanoic acid vanillylamide. “Capsinoids” mean a capsaicinoid-like substance with no pungent property, contained in Capsicum annuum L. Examples of such capsinoids include, for example, capsiate, dihydrocapsiate, nordihydrocapsiate, homocapsiate, homodihydrocapsiate and the like.

A preferable example of capsaicinoid or the like used in the present invention includes a capsaicin listed in the Pharmacopoeia of the United States of America containing about 55% by mass or more of capsaicin (C₁₈H₂₇NO₃) and the like. A more preferable example, a further more preferable example and a particularly preferable example are a capsaicinoid or the like containing about 80% by mass or more of capsaicin (C₁₈H₂₇NO₃), a capsaicinoid or the like containing about 90% by mass or more of capsaicin (C₁₈H₂₇NO₃) and a capsaicinoid or the like containing about 98% by mass or more of capsaicin (C₁₈H₂₇NO₃), respectively.

Capsaicinoids and capsinoids can be obtained by purification/isolation from plant body and/or fruits of Capsicum annuum L. containing capsaicinoids and capsinoids. The purification/isolation from Capsicum annuum L. can be performed by methods well known to those in the art such as solvent extraction, various kinds of chromatography including silica gel chromatography, preparative high performance liquid chromatography and the like, solely or in an appropriate combination thereof.

Also, capsaicin (C₁₈H₂₇NO₃) can be synthesized by known synthesis methods described in, for example, (a) E. Spath, S. F. Darling, Ber. 63, 737(1930); (b) L. Crombie, S. H. Dandegaonker, K. B. Simpson, J. Chem. Soc. 1955, 1025; (c) Osawa et al., Journal of Tohoku Pharmaceutical University, 23, 117(1976); (d) P. M. Gannett, D. L. Nagel, P. J. Reilly, T. Lawson, J. Sharpe, B. Toth, J. Org. Chem. 1988; (e) H. Kaga, M. Miura, K. Orito, J. Org. Chem. 1989, 54, 3477; (f) published unexamined Japanese patent application 1983-163699 and the like.

In addition, nordihydrocapsaicin was prepared, for example, according to the methods described in J. Chem. Research(S), 1987, pp. 344-345, and J. Org. Chem., 1989, vol. 54, pp. 3477-3478. Capsiate was prepared, for example, according to the methods described in J. Org. Chem., 1989, vol. 54, pp. 3477-3478, and Eur. J. Nutr., 2003, vol. 42, pp. 2-9. Dihydrocapsiate was prepared, for example, according to the methods described in J. Chem. Research (S), 1987, pp. 344-345, and Eur. J. Nutr., 2003, vol. 42, pp. 2-9. Nordihydrocapsiate was prepared, for example, according to the methods described in J. Chem. Research (S), 1987, pp. 344-345, J. Org. Chem., 1989, vol. 54, pp. 3477-3478, and Eur. J. Nutr., 2003, vol. 42, pp. 2-9.

Nonylic vanillylamide and decanoic acid vanillylamide which are a synthetic capsaicin are prepared, for example, according to the method described in J. Med. Chem. 1993, vol. 36, p. 2595.

The concentration of capsaicinoid or the like in the IGF-1 secretagogue of the present invention is not specifically limited as long as it permits the secretion of IGF-1. However, since capsaicinoid or the like is a substance causing strong local irritation, it is preferable that its concentration at the administration site is that causing no irritation, in consideration of such irritating property. Although an analgesic effect, etc., that is generally known as one of the effects of capsaicinoid or the like, is enhanced concentration-dependently as its concentration increases, the IGF-1 secretagogue of the present invention is characterized in that IGF-1 secretion reaches maximum within a range of concentration of capsaicinoid or the like of 0.0002 to 0.1% by mass, preferably 0.005 to 0.025% by mass in the case where, for example, the cream of the present invention is applied once. Accordingly, the concentration of capsaicinoid or the like in the IGF-1 secretagogue of the present invention is about 0.001 to 0.1% by mass, preferably about 0.005 to 0.1% by mass, and more preferably about 0.005 to 0.025% by mass.

The IGF-1 secretagogue of the present invention can increase the secretion of IGF-1 in the skin. IGF-1 is a growth factor that is similar to insulin in structure and action, and promotes differentiation, proliferation and intercellular substance productivity of cells, particularly fibroblasts, in tissues, particularly in the skin. Examples of said intercellular substance include skin collagen, elastin, hyaluronic acid and the like. Activation of differentiation or proliferation of said fibroblasts leads to production of large amounts of collagen, elastin and hyaluronic acid. Collagen and elastin are main fibers of the dermis having a function to maintain skin elasticity. Hyaluronic acid has a high water-holding capacity and helps firm the skin. Therefore, when fibroblasts produce large amounts of those intercellular substances, it is possible to firm skin and reduce fine wrinkles.

The IGF-1 secretagogue of the present invention is preferably mixed with a nonionic surfactant, an amphoteric surfactant or a cationic surfactant. Such surfactants may be used solely or in combination of two or more of them. When those surfactants are mixed in the IGF-1 secretagogue, irritation to the skin caused by capsaicinoid or the like is reduced. As a result, IGF-1 secretion is promoted at the sites where the IGF-1 secretagogue of the present invention has been applied.

Examples of nonionic surfactants include polyoxyethylene castor oil (e.g. polyoxyethylene (3) castor oil, polyoxyethylene (10) castor oil, polyoxyethylene (20) castor oil, polyoxyethylene (40) castor oil, polyoxyethylene (50) castor oil, polyoxyethylene (60) castor oil, etc.), polyoxyethylene hydrogenated castor oil (e.g. polyoxyethylene (10) hydrogenated castor oil, polyoxyethylene (40) hydrogenated castor oil, polyoxyethylene (50) hydrogenated castor oil, polyoxyethylene (60) hydrogenated castor oil, polyoxyethylene (80) hydrogenated castor oil, etc.), polyethylene glycol fatty acid ester (e.g. polyethylene glycol monolaurate, polyethylene glycol monostearate, polyethylene glycol monooleate, polyethylene glycol distearate, polyethylene glycol diisostearate, etc.), polyoxyethylene alkyl ether (e.g. polyoxyethylene (25) lauryl ether, polyoxyethylene (15) cetyl ether, polyoxyethylene (4) stearyl ether, polyoxyethylene (20) oleyl ether, etc.), polyoxyethylene polyoxypropylene alkyl ether (e.g. poloxamer 235, etc.), polyoxyethylene alkyl phenyl ether (e.g. polyoxyethylene (7.5) nonyl phenyl ether, polyoxyethylene (3) octyl phenyl ether, etc.), and polyoxyethylene sorbitan fatty acid ester (e.g. polyoxyethylene (60) sorbitan tetraoleate, polyoxyethylene (6) sorbitanmonolaurate, polyoxyethylene (6) sorbitan hexastearate, etc.).

Examples of amphoteric surfactants include alkyldimethylamino acetic acid betaines such as lauryldimethylamino acetic acid betaine, coconut oil fatty acid amide propyldimethylamino acetic acid betaine and the like.

Examples of cationic surfactants include alkyl ammonium salts such as cetyl trimethyl ammonium chloride, stearyl trimethyl ammonium chloride, dioctyl dimethyl ammonium chloride, distearyl dimethyl ammonium chloride and the like.

Among said surfactants, nonionic surfactants are preferable, polyoxyethylene hydrogenated castor oil is more preferable, and polyoxyethylene (40) hydrogenated castor oil, polyoxyethylene (50) hydrogenated castor oil or polyoxyethylene (60) hydrogenated castor oil is particularly preferable.

Although the blending amount of said surfactants depends on the kind of surfactant to be used, it is about 5% by mass or more, preferably about 9% by mass or more, and more preferably about 18% by mass or more, relative to the whole insulin-like growth factor-1 secretagogue. The upper limit of the blending amount of said surfactants is about 30% by mass, although it depends on the kind of surfactant to be used.

Although the blending ratio of capsaicin and said surfactants in the IGF-1 secretagogue of the present invention depends on the kind of surfactants to be used, the surfactant is usually used in about 50 to 30,000 parts by mass, preferably about 360 to 3,600 parts by mass, relative to capsaicinoid or the like.

An example of the IGF-1 secretagogue having the above-mentioned blending ratio includes a preparation which comprises about 0.001 to 0.1 of capsaicinoid or the like containing 98% by mass or more of capsaicin (C₁₈H₂₇NO₃), and about 5 to 18% by mass of polyoxyethylene hydrogenated castor oil.

The IGF-1 secretagogue of the present invention is preferably used as a skin external preparation. Preferable examples of the skin external preparation include drugs or quasi-drugs such as liquid preparation, suspension, emulsion, cream, ointment, gel, liniment, lotion, cataplasm and the like. The external preparations also include cosmetics such as skin-care cosmetics or cosmetics for makeup, for example, cosmetic water, cosmetic emulsion, cosmetic cream, cosmetic gel, cosmetic lotion, pack, foundation, facial cleansing agent, body soap, hand cream, shampoo, hair conditioner, hair styling product, hair growth agent and the like.

The skin external preparation of the present invention may be prepared by adding, for example, a base component and an additive mentioned below to capsaicinoid or the like and the above-mentioned surfactant, depending on the dosage form, and then mixing them. In this formulation, such preparation can be carried out according to or in a similar manner to the conventional method such as the method described in General Rules for Preparations, Japanese Pharmacopoeia, Fourteenth Edition. Further, since capsaicinoid is almost insoluble in cold water, but is easily soluble in an oil or an alcohol, it is preferable to prepare the skin external preparations of the present invention by incorporating a component capable of easily dissolving capsaicinoid as a base.

In the case of liquid preparation for external skin use of the present invention, they can be prepared by adding, for example, a solvent (e.g. normal water, purified water, normal saline solution, isopropanol, glycerine, ethanol, propylene glycol, macrogol 400, etc.; hereinafter, the same is true for the term “solvent”) to capsaicinoid or the like and the above surfactant, and mixing them for dissolution.

In the case of suspension for external skin use of the present invention, they can be prepared by adding, for example, a suspending agent/thickner (e.g. gum arabic, carmellose, carmellose sodium, carmellose calcium, hydroxypropyl cellulose, hydroxypropyl methyl cellulose, povidone, methylcellulose, aluminum monostearate, etc.; hereinafter, the same is true for the term “suspending agent/thickner”), purified water or a fatty oil (e.g. synthetic oil such as medium chain fatty acid triglyceride and hard fat, olive oil, soy bean oil, rapeseed oil, peanut oil, safflower oil, sesame oil, rice bran oil, sesame oil, camellia oil, corn oil, cottonseed oil, coconut oil, squalene, or hydrogenated oil thereof, etc.; hereinafter, the same is true for the term “fatty oil”) to capsaicinoid or the like and the above surfactant, stirring the mixture with, for example, a mixer and then homogenizing the whole.

In the case of emulsion for external skin use of the present invention, they can be prepared by adding, for example, an emulsifier (e.g. polyoxyl 40 stearate, sorbitan sesquioleate, polysorbate 80, sodium lauryl sulfate, lauromacrogol, gum arabic, cholesterol, stearic acid, glycerine monostearate, povidone, etc.; hereinafter the same is true for the term “emulsifier”) and purified water to capsaicinoid or the like and the above surfactant, stirring the mixture with, for example, a homomixer and then homogenizing the whole.

In the case of cream for external skin use of the present invention, they can be prepared by adding, for example, an oil (e.g. liquid paraffin, white vaseline, etc.), a higher alcohol (e.g. cetanol, stearyl alcohol, oleayl alcohol, cholesterol, etc.; hereinafter the same is true for the term “higher alcohol”), a water-soluble polymer (e.g. carboxyvinyl polymer, etc.), a higher fatty acid (e.g. stearic acid, behenic acid, palmitic acid, oleic acid, etc.) or a polyhydric alcohol (e.g. propylene glycol, polyethylene glycol, glycerine, 1,3-butylene glycol, etc.) and purified water to capsaicinoid or the like and the above surfactant, emulsifying the mixture, for example, under heating, and cooling the emulsion with further stirring.

In the case of ointment for external skin use of the present invention, they can be prepared by using, as a base, for example, fat (e.g. lard, beef tallow, etc.), fatty oil, lanolin, vaseline (e.g. white vaseline, yellow vaseline, etc.), paraffin, wax (e.g. beeswax, etc.), resin, plastics (e.g. microcrystalline wax, polyethylene powder, etc.), glycols, higher alcohol, glycerine, water, emulsifier, or suspending agent as it is, or using, as a base, an emulsion prepared similarly to the procedure of the above emulsion, adding capsaicinoid or the like to the said base, and then homogenizing the whole.

In the case of gel for external skin use of the present invention, they can be prepared by using as a base component, for example, water (e.g. purified water, distilled water, etc.), a higher alcohol or a gelling agent (e.g. carboxyvinyl polymer such as carbopol 941, manufactured by Noveon Inc.), dissolving capsaicinoid or the like and the above surfactant together with these bases under heating, and then cooling the solution with stirring.

In the case of liniment for external skin use of the present invention, they can be prepared by adding capsaicinoid or the like and the above surfactant to an additive including, for example, ethanol, fatty oil, glycerine, soap, emulsifier, suspending agent, etc. or a mixture thereof, and adding an refined oil such as menthol, and then making the whole homogeneously into a paste form.

In the case of lotion for external skin use of the present invention, they can be prepared by adding capsaicinoid or the like and the above surfactant to an aqueous medium together with a solvent, an emulsifier or a suspending agent, that is, for example, by admixing capsaicinoid and the above surfactant with ethanol, a fatty oil, glycerine, a soap, an emulsifier or a suspending agent, and then homogenizing the whole.

In the case of cataplasm for external skin use of the present invention, they can be prepared by admixing capsaicinoid or the like and the above surfactant with, for example, glycerine, water (e.g. purified water, distilled water, etc.), etc., adding a refined oil such as menthol, etc., and then homogenizing the whole into a paste form.

Cosmetics such as cosmetic water, cosmetic emulsion, cosmetic cream, hand cream, pack, foundation, body soap, facial cleansing agent, body soap, hand cream, shampoo, hair conditioner, hair styling agent, hair growth agent, etc. can also be prepared in a similar manner to the above-mentioned method concerning the preparation of liquid preparations, creams, lotions, liniments, etc.

A pack may take any form such as a mask for face, etc. comprising a nonwoven fabric and the like soaked in the said cosmetic water or emulsion; a peel-off type which is applied, dried, and then peeled off; and a cream type.

Components which can be used in drugs, quasi-drugs or cosmetics may be optionally added to the above-mentioned external preparations of the present invention. Examples of such components include lower alcohols (e.g. ethanol, isopropanol, etc.), refrigerants (e.g. menthol, camphor, borneol, peppermint oil, camphor, rosemary oil, etc.), wetting agents/moisturizers (e.g. hyaluronic acid, chondroitin sulfate, glycerine, D-sorbitol, propylene glycol, xylitol, sorbit, trehalose, etc.), aromas (e.g. methyl salicylate, fennel oil, orange peel tincture, dl-menthol, etc.), pH conditioners (e.g. hydrochloric acid, citric acid, acetic acid, tartaric acid, sodium hydrogen carbonate, sodium hydroxide, triethanolamine, etc.), preservatives (e.g. parahydroxybenzoic acid esters, benzyl alcohol, benzalkonium chloride, benzetonium chloride, chlorobutanol, etc.) or stabilizers (e.g. sodium edentate, ascorbic acid, sodium sulfite, etc.).

In the case where the skin external preparation of the present invention is liquid, suspension, emulsion, cream, ointment, gel, liniment or lotion, such skin external preparation as it is can be applied to the skin. In the case of cataplasm, application to the skin is possible when the skin external preparation is applied to or impregnated with a supporting material such as woven fabric or nonwoven fabric, and then a liner of polypropylene film is attached thereto.

The IGF-1 secretagogue of the present invention as a skin external preparation, for example, can be appropriately applied when it is applied about two to ten times a day in the case of a cream containing 0.001% by mass of capsaicinoid or the like, and one to several times a day, preferably one to five times a day in the case of a cream containing 0.01% by mass of capsaicinoid or the like.

### EXAMPLES

The present invention is illustrated in more detail based on the following Examples, but it is not construed to be limited to these Examples.

### Example 1

Cream:

| Liquid paraffin | 17 | g |
|---|---|---|
| White vaseline | 5 | g |
| Cetanol | 4 | g |
| Polyoxyethylene(60) hydrogenated castor oil | 18 | g |
| Triethanolamine | 0.075 | g |
| Carbopol 941 | 0.05 | g |
| Capsaicinoids containing not less than 98% by mass of | 0.01 | g |
| capsaicin (C₁₈H₂₇NO₃) | | |
| Sodium edetate | 0.1 | g |
| Methyl parahydroxybenzoate | q.s. | |
| Butyl parahydroxybenzoate | q.s. | |
| Purified water | total 100 | g |

Preparation Method:

Oily phase components which are liquid paraffin, white vaseline, cetanol, polyoxyethylene (60) hydrogenated castor oil, methyl parahydroxybenzoate, butyl parahydroxybenzoate and capsaicinoids were dissolved under heating. On the other hand, as an aqueous phase component, carbopol 941 was dispersed in distilled water, and to this dispersed solution were added triethanolamine and sodium edetate while heating. The oily phase was added to the aqueous phase with stirring to emulsify, and cooled with stirring to formulate a cream.

### Example 2

Following the procedure similar to Example 1, but using 0.005 g, 0.025 g, 0.05 g or 0.1 g of capsaicinoids containing not less than 98% by mass of capsaicin (C₁₈H₂₇NO₃), or 0.005 g, 0.01 g, 0.025 g, 0.05 g or 0.1 g of capsaicinoids containing not less than 55% by mass of capsaicin (C₁₈H₂₇NO₃), in place of using 0.01 g of capsaicinoids containing not less than 98% by mass of capsaicin (C₁₈H₂₇NO₃), a cream was obtained.

### Example 3

Following the procedure similar to Example 1, but using polyoxyethylene (10) castor oil, polyethylene glycol monostearate, polyoxyethylene (20) oleayl ether, poloxamer 235, polyoxyethylene (7.5) nonyl phenyl ether, polyoxyethylene (60) sorbitan tetraoleate, lauryldimethylaminoacetate betaine or cetyl trimethylammonium chloride, in place of using polyoxyethylene (60) hydrogenated castor oil of Example 1, a cream was obtained.

### Example 4

Ointment:

| | |
|---|---|
| Liquid paraffin | 20 g |
| White vaseline | 52 g |
| Cetanol | 9.9 g |
| Polyoxyethylene (60) hydrogenated castor oil | 18 g |
| Capsaicinoids containing not less than 98% by mass of | 0.01 g |
| capsaicin (C₁₈H₂₇NO₃) | |

Preparation Method:

Liquid paraffin, white vaseline, cetanol, polyoxyethylene (60) hydrogenated castor oil, and capsaicinoid were added and kneaded under heating to prepare an ointment.

### Example 5

Following the procedure similar to Example 4, but using 0.005 g, 0.025 g, 0.05 g or 0.1 g of capsaicinoids containing not less than 98% by mass of capsaicin (C₁₈H₂₇NO₃), or 0.005 g, 0.01 g, 0.025 g, 0.05 g or 0.1 g of capsaicinoids containing not less than 55% by mass of capsaicin (C₁₈H₂₇NO₃), in place of using 0.01 g of capsaicinoids containing not less than 98% by mass of capsaicin (C₁₈H₂₇NO₃), an ointment was obtained.

### Example 6

Following the procedure similar to Example 4, but using polyoxyethylene (10) hydrogenated castor oil, polyethylene glycol monostearate, polyoxyethylene (20) oleayl ether, poloxamer 235, polyoxethylene (7.5) nonyl phenyl ether, polyoxyethylene (60) sorbitan tetraoleate, lauryldimethylaminoacetate betaine or cetyltrimethylammonium chloride, in place of using polyoxyethylene (60) hydrogenated castor oil, an ointment was obtained.

### Example 7

Gel:

| | |
|---|---|
| Polyoxethylene (60) hydrogneated castor oil | 9.0 g |
| Carbopol 941 | 0.5 g |
| Ethanol | 22 g |
| Triethanolamine | 1.5 g |
| Capsaicinoids containing not less than 55% by mass of | 0.025 g |
| capsaicin (C₁₈H₂₇NO₃) | |
| Purified water | 57.9 g |

Preparation Method:

Polyoxyethylene (60) hydrogenated castor oil and carbopol 941 were added to distilled water to dissolve under heating, and to this solution was added a solution of capsaicinoid dissolved by addition of ethanol. The solution was stirred and triethanolamine was added thereto, followed by cooling with stirring to obtain a gel.

### Example 8

Following the procedure similar to Example 7, but using 0.005 g, 0.01 g, 0.025 g, 0.05 g or 0.1 g of capsaicinoids containing not less than 98% by mass of capsaicin (C₁₈H₂₇NO₃), or 0.005 g, 0.01 g, 0.05 g or 0.1 g of capsaicinoids containing not less than 55% by mass of capsaicin (C₁₈H₂₇NO₃), in place of using 0.025 g of capsaicinoids containing not less than 55% by mass of capsaicin (C₁₈H₂₇NO₃), a gel is obtained.

### Example 9

Following the procedure similar to Example 7, but using polyoxyethylene (10) castor oil, polyethylene glycol monostearate, polyoxyethylene (20) oleayl ether, poloxamer 235, polyoxyethylene (7.5) nonyl phenyl ether, polyoxyethylene (60) sorbitan tetraoleate, lauryldimethylaminoacetate betaine or cetyltrimethylammonium chloride, in place of using polyoxyethylene (60) hydrogenated castor oil as in Example 7, a gel was obtained.

### Example 10

Liquid Preparation:

| | |
|---|---|
| Polyoxethylene (60) hydrogenated castor oil | 12 g |
| Ethanol | 22 g |
| Capsaicinoids containing not less than 55% by mass of | 0.01 g |
| capsaicin (C₁₈H₂₇NO₃) | |
| Purified water | 59.9 g |

Preparation Method:

Capsaicinoids were dissolved in ethanol, and to this solution were added polyoxyethylnene (60) hydropgenated castor oil and purified water. The mixture was dissolved to obtain a liquid preparation.

### Example 11

Following the procedure similar to Example 10, but using 0.005 g, 0.01 g, 0.025 g, 0.05 g or 0.1 g of capsaicinoids containing not less than 98% by mass of capsaicin (C₁₈H₂₇NO₃), or 0.005 g, 0.025 g, 0.05 g or 0.1 g of capsaicinoids containing not less than 55% by mass of capsaicine (C₁₈H₂₇NO₃), in place of using 0.01 g of capsaicinoids containing not less than 55% by mass of capsaicin, there was prepared a liquid preparation.

### Example 12

Following the procedure similar to Example 10, but using polyoxyethylene (10) castor oil, polyethylene glycol monostearate, polyoxyethylene (20) oleayl ether, poloxamer 235, polyoxethylene (7.5) nonyl phenyl ether, polyoxyethylene (60) sorbitan tetraoleate, lauryldimethylaminoacetate betaine or cetyltrimethylammonium chloride, in place of using polyoxyethylene (60) hydrogenated castor oil in Example 10, there was prepared a liquid preparation.

### Example 13

Cataplasm:

| | |
|---|---|
| Polyoxyethylene (60) hydrogenated castor oil | 18 g |
| Glycerine | 10 g |
| Carboxymethylcellulose sodium | 50 g |
| Capsaicinoids containing not less than 55% by mass of | 0.0005 g |
| capsaicin (C₁₈H₂₇NO₃) | |
| Purified water | 21.9 g |

Preparation Method:

Water, polyoxyethylene (60) hydrogenated castor oil and carboxymethylcellulose sodium were kneaded well under heating, and to this mixture was added a solution of capsaicinoids dissolved in glycerine. The mixture was kneaded well and then cooled to prepare a cataplasm.

### Example 14

Following the procedure similar to Example 13, but using 0.005 g, 0.01 g, 0.025 g, 0.05 g, or 0.1 g of capsaicinoids containing not less than 98% by mass of capsaicin (C₁₈H₂₇NO₃), or 0.01 g, 0.025 g, 0.05 g or 0.1 g of capsaicinoids containing not less than 55% by mass of capsaicin (C₁₈H₂₇NO₃), in place of using 0.005 g of capsaicinoids containing not less than 55% by mass of capsaicin ((C₁₈H₂₇NO₃), there was prepared a cataplasm.

### Example 15

Following the procedure similar to Example 13, but using polyoxyethylene (10) castor oil, polyethylene glycol monostearate, polyoxyethylene (20) oleayl ether, poloxamer 235, polyoxyethylene (7.5) nonyl phenyl ether, polyoxyethylene (60) sorbitan tetraoleate, lauryldimethylaminoacetate betaine or cetyltrimethylammonium chloride, in place of using polyoxyethylene (60) hydrogenated castor oil in Example 13, there was prepared a cataplasm.

### Test Example 1

Activating action of IGF-1 on mouse skin (See FIG. 1) Animals used: male C57BL/6 mice of 8-weeks of age were used. One group consisted of three mice.

Cream tested: a cream of Example 1, and a cream prepared in a similar manner to Example 1, except that each preparation contained 0.001% by mass, 0.005% by mass, 0.025% by mass or 0.1% by mass of the capsaicinoids, were used. The base preparation is a preparation of Example 1 from which capsaicinoids are excluded.

Test Method:

Mice were anesthetized by intraabdominal administration of ketaral (100 mg/kg) and xylazine (10 mg/kg). A shaving cream was applied to the mouse back skin, and hair was removed with a razor. Each of the test creams or the base preparation was applied in an amount of 100 mg per 1 cm² to the area of mouse back skin where hair had been removed. The skin sampling at the application site was performed 30 minutes after the application of the test creams or the base preparation. The skin samples (each 1 cm²) were freeze-dried with a liquid nitrogen gas, homogenized with IN acetic acid and preserved at −80° C. until the determination of IGF-1. The determination of IGF-1 in said frozen and preserved homogenate was performed by EIA method using an IGF-1 determination kit (ACTIVE, registered trade mark, rat IGF-1 EIA; manufactured by Cosmo Bio Co., Ltd.), and the measured value was regarded as the amount of IGF-1 contained in the mouse back skin.

The amount (level) of IGF-1 on the mouse back skin was shown in FIG. 1. When each cream containing 0.005%, 0.01%, 0.025%, or 0.1% by mass of capsaicinoids was applied to the mouse back skin, the content of IGF-1 in the mouse skin was significantly elevated in comparison with the skin where the base preparation was applied, showing that the IGF-1 content in the skin reached a peak when a cream containing 0.01% by mass of capsaicinoids was applied.

### Test Example 2

Before test, a picture of wrinkles at the corner of the eyes of a male panelist (50 years old) was taken. The cream of Example 1 was applied to the corner of the eyes two times per day for 5 days. After 5 days, a picture of wrinkles was taken in the same way as before the test in the same direction and under the same light quantity. Comparison between the pictures of before the test and after 5 days of the application of the cream of Example 1 showed that the wrinkles at the corner of the eyes became less visible (see FIG. 2).

### Test Example 3

Action of various capsaicinoids on mouse IGF-1 Animals used: male C57BL/6 mice of 7- to 10-weeks of age were used. One group consisted of three mice.

Test Drugs:

Capsaicin (purity: 97%), dihydrocapsaicin, nordihydrocapsaicin, capsiate, dihydrocapsiate, nordihydrocapsiate, and nonylic vanillylamide were used as test drugs.

After each 5 mg of test drugs were weighed, they were respectively dissolved in 1 mL of ethanol [99.5% v/v; product of Wako Pure Chemical Industries, Ltd.]. Each solution was diluted with physiological saline solution (49 mL) to a total volume of 50 mL (final concentration of each test drug: 0.01% by mass). The resulting solution was served as a test solution. As a base preparation (control), a mixed solution of ethanol (1 mL) and physiological saline solution (49 mL) was used.

Herein, capsaicin (purity: 97%) and dihydrocapsaicin were available from Sigma-Aldrich Co. Nordihydrocapsaicin was prepared according to the methods described in J. Chem. Research(S), 1987, pp. 344-345, and J. Org. Chem., 1989, vol. 54, pp. 3477-3478. Capsiate was prepared according to the methods described in J. Org. Chem., 1989, vol. 54, pp. 3477-3478, and Eur. J. Nutr., 2003, vol. 42, pp. 2-9. Dihydrocapsiate was prepared according to the methods described in J. Chem. Research (S), 1987, pp. 344-345, and Eur. J. Nutr., 2003, vol. 42, pp. 2-9. Nordihydrocapsiate was prepared according to the methods described in J. Chem. Research (S), 1987, pp. 344-345, J. Org. Chem., 1989, vol. 54, pp. 3477-3478, and Eur. J. Nutr., 2003, vol. 42, pp. 2-9. Nonylic vanillylamide was available from Wako Pure Chemical Industries, Ltd.

Test Method:

A test was performed in a similar manner to Test Example 1, except that each test solution was applied in an amount of 0.2 mL/cm² to the mouse back skin in place of 100 mg/cm² of the test cream or the base preparation.

The amount (level) of IGF-1 on the mouse back skin to which each test solution was applied was shown in FIG. 3 and FIG. 4. When each test solution containing 0.01% by mass of capsaicin (purity: 97%), dihydrocapsaicin, nonylic vanillylamide, nordihydrocapsaicin, capsiate, dihydrocapsiate, or nordihydrocapsiate nonylic vanillylamide, was applied to the mouse back skin, the amount of IGF-1 in the mouse back skin was significantly elevated compared to that in the mouse back skin where the base preparation was applied.

This result proves that any of capsaicinoids, capsinoids and synthetic capsaicin have an action of increasing the level of skin IGF-1.

### INDUSTRIAL APPLICABILITY

The present invention has been developed on the basis of a clear view about the action mechanism of pharmaceutical effect of capsaicinoids or the like, and resides in that the blending amount of capsaicinoid or the like is small and, nonetheless, its pharmaceutical effect is large. Therefore, problems such as skin irritation, etc. occurred when capsaicinoid or the like is applied to the skin as a skin external preparation are also solved. As a result, the present invention provides a useful insulin-like growth factor-1 secretagogue containing capsaicinoid or the like which exerts sufficient pharmaceutical effects such as promotion of alleviation of skin wrinkles and sagging skin due to, for example, increase of skin thickness and elasticity, and promotion of hair growth.

### BRIEF EXPLANATION OF THE DRAWINGS

FIG. 1 is a view showing the relation between capsaicinoid concentration in the skin external preparation applied and IGF-1 content in mouse skin. In the figure, * shows the significant difference (p<0.01) to the base preparation.

FIG. 2 is a view showing the change before and after application of the cream of Example 1.

FIG. 3 is a view showing the IGF-1 content in the mouse skin after capsaicin (purity: 97%), dihydrocapsaicin, nordihydrocapsaicin, capsiate, dihydrocapsiate or nordihydrocapsiate was applied to the mouse skin. In the FIG. 3, * shows the significant difference (p<0.01) to the base preparation.

FIG. 4 is a view showing the IGF-1 content in the mouse skin after capsaicin (purity: 97%) or nonylic vanillylamide was applied to the mouse skin. In the FIG. 4, * shows the significant difference (p<0.01) to the base preparation.

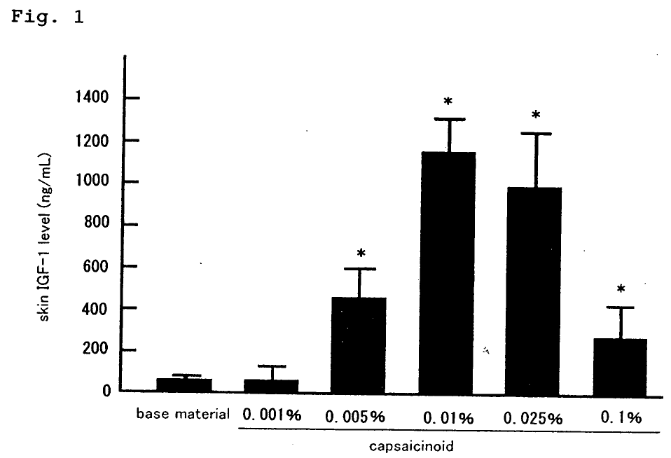

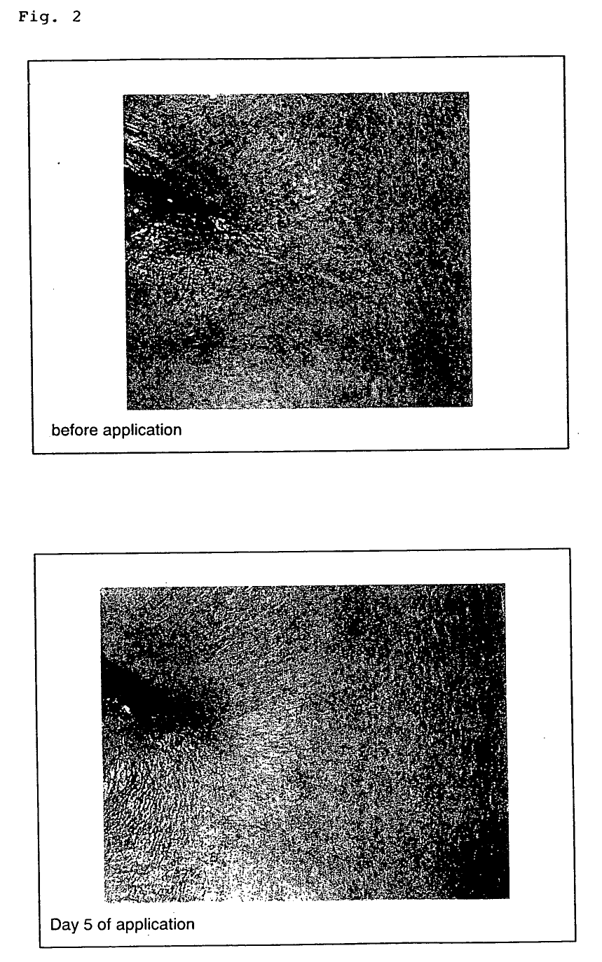

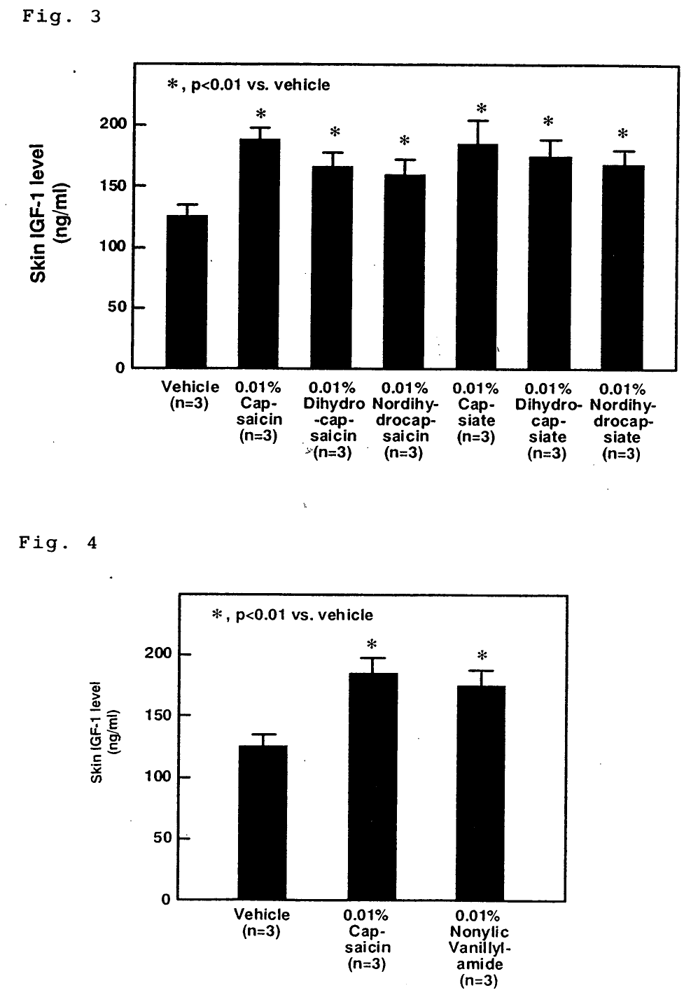

1. An insulin-like growth factor-1 secretagogue containing one or two or more compounds selected from capsaicinoids, capsinoids and synthetic capsaicin.

2. The insulin-like growth factor-1 secretagogue as claimed in claim 1, wherein the compound is contained in an amount of 0.001 to 0.1% by mass.

3. The insulin-like growth factor-1 secretagogue as claimed in claim 1, which promotes differentiation, proliferation and intercellular substance productivity of skin fibroblasts.

4. The insulin-like growth factor-1 secretagogue as claimed in claim 3, wherein the intercellular substance is one or two or more members selected from the group consisting of skin collagen, skin elastin and skin hyaluronic acid.

5. The insulin-like growth factor-1 secretagogue as claimed in claim 1, wherein capsaicinoids contain at least one compound selected from the group consisting of (6E)-N-[(4-hydroxy-3-methoxyphenyl)methyl]-8-methyl-6-nonenamide, dihydrocapsaicin, nordihydrocapsaicin, homocapsaicin, and homodihydrocapsaicin.

6. The insulin-like growth factor-1 secretagogue as claimed in claim 1, wherein capsinoids contain at least one compound selected from the group consisting of capsiate, dihydrocapsiate, nordihydrocapsiate, homocapsiate and homodihydrocapsiate.

7. The insulin-like growth factor-1 secretagogue as claimed in claim 1, wherein synthetic capsaicin is nonylic vanillylamide or decanoic acid vanillylamide.

8. The insulin-like growth factor-1 secretagogue as claimed in claim 5, wherein capsaicinoids contain 55% by mass or more of (6E)-N-[(4-hydroxy-3-methoxyphenyl)methyl]-8-methyl-6-nonenamide.

9. The insulin-like growth factor-1 secretagogue as claimed in claim 5, wherein capsaicinoids contain 98% by mass or more of (6E)-N-[(4-hydroxy-3-methoxyphenyl)methyl]-8-methyl-6-nonenamide.

10. The insulin-like growth factor-1 secretagogue as claimed in claim 3, which is a skin external preparation.

11. The insulin-like growth factor-1 secretagogue as claimed in claim 10, which further contains one or two or more compounds selected from the group consisting of nonionic surfactant, amphoteric surfactant and cationic surfactant at a rate of 5% by mass or more, relative to the whole insulin-like growth factor-1 secretagogue.

12. The insulin-like growth factor-1 secretagogue as claimed in claim 11, wherein the nonionic surfactant contains one or two or more compounds selected from the group consisting of polyoxyethylene castor oil, polyoxyethylene hydrogenated castor oil, polyethylene glycol fatty acid ester, polyoxyethylene alkyl ether, polyoxyethylene polyoxypropylene alkyl ether, polyoxyethylene alkylphenyl ether and polyoxyethylene sorbitan fatty acid ester.

13. The insulin-like growth factor-1 secretagogue as claimed in claim 11, wherein the amphoteric surfactant is an alkyldimethylamino acetic acid betaine.

14. The insulin-like growth factor-1 secretagogue as claimed in claim 11, wherein the cationic surfactant is an alkyl ammonium salt.

15. The insulin-like growth factor-1 secretagogue as claimed claim 10, which is in the dosage form of liquid preparation, suspension, emulsion, ointment, cream, gel, liniment, lotion, cataplasm or pack.

16. The insulin-like growth factor-1 secretagogue as claimed in claim 15, which comprises 0.001 to 0.1% by mass of capsaicinoids containing 98% by mass or more of (6E)-N-[(4-hydroxy-3-methoxyphenyl)methyl]-8-methyl-6-nonenamid and 5 to 18% by weight of polyoxyethylene hydrogenated castor oil.
